(19) United States
(12) Patent Application Publication
(10) Pub. No.: US 2012/0045523 A1
(43) Pub. Date: Feb. 23, 2012

(54) COMBINATION OF A TLR3 LIGAND AND A CHEMOTHERAPY AGENT WHICH ACTS ON THE INTRINSIC "APOPTOSIS" PATHWAY IN THE TREATMENT OF CANCER

(75) Inventors: Serge Lebecque, Civrieux D'Azergues (FR); Charles Dumontet, Venissieux (FR); Yves Pacheco, Charly (FR); Claire Rodriguez-Lafrasse, Venissieux (FR); Florent Toscano, Lyon (FR); Yann Estornes, Lyon (FR); Francois Virard, Lyon (FR); Isabelle Coste-Invernizzi, Chazay D'Azergues (FR); Toufic Renno, Civrieux D'Azergues (FR)

(73) Assignees: UNIVERSITE CLAUDE BERNARD LYON I, VILLEURBANNE CEDEX (FR); HOSPICES CIVILS DE LYON, LYON CEDEX 02 (FR)

(21) Appl. No.: 13/056,341

(22) PCT Filed: Jul. 31, 2009
(86) PCT No.: PCT/FR09/51543

§ 371 Date: Oct. 4, 2011

(30) Foreign Application Priority Data
Jul. 31, 2008 (FR) 0855304

(51) Int. Cl. A61K 33/24; A61P 35/00; A61K 31/7048; A61K 31/713; A61K 31/704

(52) U.S. Cl. 424/649; 514/44 R; 514/34; 514/27

(57) ABSTRACT

The present invention relates to a drug comprising separately or together (i) a TLR3 ligand and (ii) a chemotherapeutic agent that acts on the intrinsic apoptotic pathway, for simultaneous or sequential administration in the treatment of cancer, wherein the chemotherapeutic agent is selected from topoisomerase II inhibitors, platinum-derived alkylating agents and PI3 kinase inhibitors.

The present invention relates to the field of drugs for the treatment of cancer. More precisely, the present invention relates to the combination of a TLR3 ligand and a chemotherapeutic or radiotherapeutic agent that acts on the intrinsic apoptotic pathway resulting in a synergistic effect in the context of the treatment of cancer.

Treatments in the fight, against cancer are the subject of active research. Application WO2006/014653 demonstrated that Toll-like receptor 3 (TLR3) is a therapeutic target in the treatment of cancer. As mentioned in application WO2006/014653, the family of TLRs includes highly conserved protein receptors, designated TLR1 to TLR10. These human TLRs are type I transmembrane proteins that comprise a danger-signaling extracellular receptor domain and are composed of many leucine-rich repeat (LRR) motifs, a transmembrane domain and an intracellular domain containing a death domain which enables transduction of the activation signal.

Although mammalian TLRs have a large number of common characteristics and conserved signal transduction mechanisms, their biological functions are quite different. When a TLR is activated it selects a molecule, called an adaptor, to propagate the signal via its death domain. Five TLR-family adaptors are known: MyD88, TIRAP (also called MAL), TRIF, TRAM and SARM. The various biological functions are strongly related to the fact that these five different adaptors exist in various combinations with TLRs and are mediators of various types of signaling. Moreover, TLRs are expressed differently in hematopoietic and nonhematopoietic cells. Consequently, the response of a TLR ligand depends both on the type of TLR signaling and on the nature of the cells in which the TLR is expressed.

The sequence of human TLRs 1 to 10 is described in patent application WO01/90151, although the sequence of these proteins is named differently compared to the public nomenclature. The TLR3 nucleotide sequence and amino acid sequence can be accessed in the GenBank database under numbers NM003265 and NP003256, respectively.

The expression of TLR receptors by human cancer cells led to the study of the effects of their ligands on tumor growth. In particular, the effects of TLR3 activation on breast cancer cell lines (Salaun B et al.: TLR3 can directly trigger apoptosis in human cancer cells, J Immunol 2006, 176:4894-4901), melanoma cell lines (Salaun et al.: Toll-like receptor 3 expressed by melanoma cells as a target for therapy?, Clin Cancer Res 2007, 13:4565-4574), myeloma cell lines (Jego et al.: Pathogen-associated molecular patterns are growth and survival factors for human myeloma cells through Toll-like receptors, Leukemia 2006, 20:1130-1137), hepatoma cell lines (Khvalevsky et al.: TLR3 signaling in a hepatoma cell line is skewed towards apoptosis, J Cell Biochem 2007, 100:1301-1312), clear cell renal cell carcinoma cell lines (Morikawa et al.: Identification of Toll-like receptor 3 as a potential therapeutic target in clear cell renal cell carcinoma, Clin Cancer Res 2007, 13:5703-5709), prostate cancer cell lines (Paone et al.: Toll-like receptor 3 triggers apoptosis of human prostate cancer cells through a PKC-α dependent mechanism, Carcinogenesis 2008), glioma cell lines and mesothelioma cell lines (unpublished observations) were analyzed.

Several TLR3 ligands are known, notably viral and synthetic double-stranded RNA, such as polyinosinic-polycytidylic acid (poly(I:C)), polyadenylic-polyuridylic acid (poly(A:U)) and a modified form (polyI:polyC₁₂U) (Carter et al.: Comparative studies of ampligen (mismatched double-stranded RNA) and interferons, J Biol Response Mod 1985, 4:613-620) which are ligands of high molecular weight and of heterogeneous size (Alexopoulou et al.: Recognition of double-stranded RNA and activation of NF-kappaB by Toll-like receptor 3, Nature 2001, 413:732-738).

In the context of the invention, the Inventors have shown that TLR3 ligands that act by caspase 8, and thus by the extrinsic apoptotic pathway, lead, in combination with agents acting by the intrinsic pathway, to a synergistic effect, notably on NSCLC cell apoptosis. The results presented below show the synergies obtained with various radiotherapy agents or chemotherapy agents of distinct classes, and are thus highly reproducible.

The prior publications cited below by no means made it possible to envisage such a combination, in the context of a therapeutic treatment:
Document D1 by Kovark J. et al. (Neoplasma, 1977, NLM270616) describes the effectiveness of the combination of poly(I:C) with cisplatin in an in vivo model of rat myelogenous leukemia (RBA-Le cell line). Nevertheless, in the experimental context described, it must be taken into account that poly(I:C) activates TLR3, but also intracellular receptors RIG-I and MDA-5. Moreover, the sensitivity of the RBA-Le cell line to TLR3 activation (apoptosis) is not established, and relatively improbable according to the results obtained with mouse tumors. The action of poly(I:C) is thus in all likelihood independent of apoptosis, and the synergy with platinum salt likely results from the pro-apoptotic effect of platinum salt on the tumor and from the immunostimulatory effect of poly(I:C). In this context, the murine model cannot be regarded as a reliable model whose results can be applied to man. The teaching of this document by no means describes a method of therapeutic treatment in man, nor a drug that integrates, in addition to cisplatin, a TLR3 ligand.
Document WO 2007/144985 proposes to combine RPN2 gene expression inhibitor siRNAs with chemotherapy agents, notably docetaxel or cisplatin. Crystallographic analysis of human TLR3 protein bound with its ligand suggests that TLR3 receptor activation results from multimerization of the receptor following its binding with a double-stranded RNA longer than 48 base pairs (Lin Liu, et al.: Structural basis of Toll-like receptor 3 signaling with double-stranded RNA, Science (New York, N.Y.), 320 (2008), 379-81). From this observation, siRNAs longer than roughly 21 base pairs can be excluded as possible TLR3 ligands.

Moreover, the synergistic effect demonstrated in the context of the present invention was by no means obvious, considering the fact that:
the essential role of the extrinsic apoptotic pathway in the induction of cancer cell death by TLR3 activation had not been formally shown;
numerous TLRs (including TLR3) are able to activate the transcription of survival factors in many cell types (Jego et al.: Pathogen-associated molecular patterns are growth and survival factors for human myeloma cells through Toll-like receptors, Leukemia 2006, 20:1130-1137; Hassan et al.: TLR9 expression and function is abolished by the cervical cancer-associated human papillomavirus type 16, J Immunol 2007, 178:3186-3197; Bsibsi et al.: Identification of soluble CD14 as an endogenous agonist for Toll-like receptor 2 on human astrocytes by genome-scale functional screening glial cell derived proteins, Glia 2007, 55:473-482);
recent scientific reviews express doubts on the possibility of combining TLR ligands with chemotherapy or with radiotherapy to treat cancer (Kelly et al.: TLR-4 signaling promotes tumor growth and paclitaxel chemoresistance in ovarian cancer, Cancer Res 2006, 66:3859-3868; Huang et al.: Listeria monocytogenes promotes tumor growth via tumor cell Toll-like receptor 2 signaling, Cancer Res 2007, 67:4346-4352; Chen et al.: Regulation of IKKbeta by miR-199a affects NF-kappaB activity in ovarian cancer cells, Oncogene 2008).

In this context, the invention relates to a drug comprising separately or together (i) a TLR3 ligand and (ii) a chemotherapeutic agent that acts on the intrinsic apoptotic pathway selected from topoisomerase II inhibitors, platinum-derived alkylating agents and PI3 kinase inhibitors, for simultaneous or sequential administration in the treatment of cancer.

Advantageously, the inventive drug comprises successive administration of (i) an agent that acts on the intrinsic apoptotic pathway selected from topoisomerase II inhibitors, platinum-derived alkylating agents and PI3 kinase inhibitors and then (ii) a TLR3 ligand in the treatment of cancer.

More precisely, this drug is intended for the treatment of squamous cell lung cancer, colon adenocarcinoma, mesothelioma, glioma, breast adenocarcinoma, melanoma, clear cell kidney cancer, prostate cancer, hepatocellular carcinoma or multiple myeloma.

According to a preferred embodiment which leads to a large synergistic effect, the chemotherapeutic agent is a platinum-derived alkylating agent, for example selected from cisplatin and oxaliplatin.

According to another preferred embodiment which leads to a large synergistic effect, the chemotherapeutic agent is a topoisomerase II inhibitor, for example selected from etoposide and doxorubicin.

According to another preferred embodiment which leads to a large synergistic effect, the chemotherapeutic agent is a PI3 kinase inhibitor, for example selected from wortmannin, LY294002, PIK-90/BAY2-47, XL765, XL147, SF1126, NVP-BEZ235, NVP-BGT226, GDC-0941, CAL-101 and GSK1059615.

Advantageously, the TLR3 ligand used in combination with the chemotherapeutic agents above is a TLR3 agonist, notably a synthetic double-stranded RNA, such as poly(I:C) or a specific TLR3 ligand such as poly(A:U).

The present invention thus relates, according to a particular embodiment, to a drug comprising separately or together (i) a synthetic double-stranded RNA TLR3 ligand, in particular an agonist, such as poly(I:C), and (ii) a platinum-derived alkylating agent, for example selected from cisplatin and oxaliplatin, or a topoisomerase II inhibitor, for example selected from etoposide and doxorubicin, or a PI3 kinase inhibitor, for example selected from wortmannin, LY294002, PIK-90/BAY2-47, XL765, XL147, SF1126, NVP-BEZ235, NVP-BGT226, GDC-0941, CAL-101 and GSK1059615, for simultaneous or sequential administration in the treatment of cancer.

According to a particular embodiment of the invention, the drug is provided in the form of a single pharmaceutical composition combining, in the same formulation, (i) a TLR3 ligand and (ii) a chemotherapeutic agent that activates the intrinsic apoptotic pathway selected from topoisomerase II inhibitors, platinum-derived alkylating agents and PI3 kinase inhibitors.

The invention thus also relates to the use of a TLR3 ligand and a chemotherapeutic agent that acts on the intrinsic apoptotic pathway selected from topoisomerase II inhibitors, platinum-derived alkylating agents and PI3 kinase inhibitors for the preparation of a drug as defined above.

Treatment methods in human beings corresponding to the administration of such a drug also form an integral part of the invention.

### DEFINITIONS

“Ligand” refers to any molecule able to bind specifically to another molecule or to a receptor. “Ligand” includes both agonists and antagonists. A TLR3 ligand is a molecule or a combination of molecules able to lead to the multimerization of TLR3 and/or the conformation change necessary to activate the signaling pathway controlled by TLR3.

A ligand can be, for example, a small organic molecule, an antibody or an antibody fragment, an oligonucleotide or a modified oligonucleotide, a polypeptide, a DNA or an RNA. From the nucleic acid and amino acid sequences of TLR3, the person skilled in the art are able to produce an antibody that recognizes the protein, an oligonucleotide or a modified oligonucleotide, a polypeptide, a DNA or an RNA, according to standard molecular biology techniques. Notably, synthetic double-stranded RNA TLR3 ligands, as described on pages 20 to 26 of the patent application WO2006/054177, are preferred in the context of the invention. As a nonrestrictive example, the synthetic dsRNAs poly(A:U) and poly(I:C) sold by Invivogen can be cited.

“Agonist” refers to a ligand able to bind to and to activate a receptor. Further details on the TLR3 agonists that can be used in the context of the invention are contained in the patent application WO2006/054177, incorporated by reference. TLR3 agonists can be identified by the demonstration of their direct or indirect binding to the receptor (for example, by biochemical, microscopy or flow cytometry techniques), and by the demonstration of their ability to activate, in cells expressing functional TLR3, at least one of the biological functions triggered by TLR3: production of inflammatory cytokines, production of type I interferon, activation of NF-κB and activation of p38 and JNK MAPKs (Uematsu et al.: Toll-like receptors and Type I interferons, J Biol Chem 2007, 282:15319-15323). TLR3 agonists will be notably characterized by a cytokine concentration or a transcription activation level greater than the values observed with non-activated cells plus two standard deviations.

“Specific TLR3 ligand” refers to a ligand that is recognized only by the TLR3 membrane receptor, and not by intracellular receptors such as RIG-I, MDA-5 and PKR. Examples of such a ligand includes the ligand poly(A:U) or specific synthetic double-stranded RNA, in contrast with poly(I:C) whose activity is based not only on its interaction with TLR3 but also by intracellular receptors, whereas poly(A:U) acts specifically on TLR3. However, in the context of the invention, the Inventors have also shown that the apoptotic activity of poly(I:C) depends exclusively on TLR3 because:
inhibition (by siRNA) of the expression of RIG-I, MDA-5 and PKR has no effect on poly(I:C) under the experimental conditions of the invention,
inhibition (by siRNA) of TLR3 or TRIF (the only TLR3 signaling adaptor molecule) inhibits apoptosis induced by poly(I:C),
   and that poly(A:U) triggers apoptosis of cells sensitive to poly(I:C).

“Antagonist” refers to a ligand able to bind to and to prevent the activation of a receptor. Alternatively, an antagonist can bind to an agonist of the receptor and thus prevent it from binding to a receptor. TLR3 antagonists thus defined are able to block the activation of at least one of the biological functions triggered by a TLR3 agonist.

“Apoptosis” refers to programmed cell death.

“Agent that activates the intrinsic apoptotic pathway” refers to agents that directly or indirectly activate the mitochondria-dependent apoptotic pathway, as can be established by showing the protective role of the combined overexpression of molecules Bcl-2 and Bcl-XL (Galluzzi et al.: Methods for the assessment of mitochondrial membrane permeabilization in apoptosis, Apoptosis 2007, 12:803-813).

“Chemotherapeutic agent” refers to any chemical molecule used in the treatment of cancer.

In the context of the invention, as a chemotherapeutic agent that acts on the intrinsic apoptotic pathway, an agent selected from topoisomerase II inhibitors, platinum-derived alkylating agents and PI3 kinase inhibitors is used.

Platinum-derived alkylating agents, topoisomerase II inhibitors and PI3 kinase inhibitors lead to greater synergistic effects than other chemotherapeutic agents acting on the intrinsic apoptotic pathway. Indeed, the choice of topoisomerase inhibitor, platinum-derived alkylating agent or PI3 kinase inhibitor is not arbitrary, since another class of chemotherapeutic agents, namely antimetabolites such as gemcitabine and 5-fluorouracil, led to little or no synergistic effect, and even have an antagonist effect, as shown in the examples below.

“Platinum-derived alkylating agent” refers to molecules able to bind to DNA covalently via a platinum atom. Examples include oxaliplatin, cisplatin and carboplatin.

“Topoisomerase II inhibitor” refers to a molecule able to prevent the functioning of the topoisomerase II enzyme which changes the topology of the DNA molecule and controls the twisting and winding of the two strands of the molecule. Topoisomerase activity is demonstrated by the appearance of a high molecular weight complex formed from double-stranded circular DNA in the presence of the enzyme and ATP. These complexes are revealed by a slower migration speed of the DNA in a gel or are directly observed by electron microscopy (Goto et al.: Cloning of yeast TOP1, the gene encoding DNA topoisomerase I, and construction of mutants defective in both DNA topoisomerase I and DNA topoisomerase II, Proc Natl Acad Sci U.S.A. 1985, 82:7178-7182). Examples of topoisomerase II inhibitors include etoposide, tenoposide, doxorubicin and Adriamycin.

“PI3 kinase inhibitor” refers to an inhibitor of phosphatidylinositol 3-kinase (PI3 kinase) which inhibits the PI3K/AKT kinase (or protein kinase B) signaling pathway and thus has antineoplastic activity by increasing mitochondrial membrane permeability and apoptosis. PI3 kinase inhibitors are, generally, compounds that interfere with the binding of ATP in the PI3 kinase ATP binding site, thus preventing more or less specifically the activity of these kinases. In certain cases, PI3 kinase inhibitors are allosteric inhibitors. The following publications describe PI3 kinase inhibitors (more or less specific for PI3 kinase) under development in cancer research: Romina Marone et al.: Targeting phosphoinositide 3-kinase: moving towards therapy, Biochimica Et Biophysica Acta, 1784 (2008), 159-185 and Saskia Brachmann et al.: PI3K and mTOR inhibitors: a new generation of targeted anticancer agents, Current Opinion in Cell Biology, 21 (2009), 194-198. Notably, in the context of the invention, the PI3 kinase inhibitors described in table 2 of the publication by Romina Marone et al.: Targeting phosphoinositide 3-kinase: moving towards therapy, Biochimica Et Biophysica Acta, 1784 (2008), 159-185, can be used. Examples of PI3 kinase inhibitors include wortmannin, LY294002 (Lilly), PIK-90/BAY2-47 (Bayer), XL765 and XL147 (Exelixis), SF1126 (Semafore; Cancer res. 2008, 68, 206-215), NVP-BEZ235 (Mol. Cancer. Ther., 2008, 7, 1851-1863) and NVP-BGT226 (Novartis), GDC-0941 (Genentech; J. Med. Chem., 2008, 51, 5522-5532), CAL-101 (Calistoga Pharmaceuticals) and GSK1059615 (GlaxoSmithKline), whose structural formulas are given in table 2 of the publication by Romina Marone et al.: Targeting phosphoinositide 3-kinase: moving towards therapy, Biochimica Et Biophysica Acta, 1784 (2008), 159-185, which can be referred to for further details.

“Cancer” refers to any pathological condition typically characterized by unregulated cell growth. Examples of cancer include carcinoma, lymphoma, blastoma, sarcoma and leukemia, and more precisely squamous cell lung cancer, colon adenocarcinoma, mesothelioma, glioma, breast adenocarcinoma, melanoma, clear cell kidney cancer, prostate cancer, hepatocellular carcinoma and multiple myeloma.

“Treatment” refers to any therapeutic measure that prevents or suppresses a disease or disorder leading to a desirable clinical effect or to any beneficial effect, notably including the suppression or the reduction of one or more symptoms and the regression, the slowing or the ceasing of the progression of the cancer or disorder associated with the symptoms. Such a treatment applies exclusively to humans.

“Therapeutically effective quantity” refers to any quantity of a composition that improves one or more of the characteristic parameters of cancer.

The two treatments, with the TLR3 ligand and with the chemotherapeutic agent, can be simultaneous or sequential. The two active ingredients, namely the TLR3 ligand and the chemotherapeutic agent that acts on the intrinsic apoptotic pathway, used in combination in the context of the invention, can be administered separately, each in a distinct pharmaceutical composition, in which case the administration can be simultaneous or sequential, or can be administered jointly in a single pharmaceutical composition, in which case the administration is simultaneous.

Various orders of administration can be envisaged in the context of sequential administration. The TLR3 ligand can be administered before (for example, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or after (for example, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) administration of the chemotherapeutic agent or irradiation.

Preferably, administration of the TLR3 ligand and the chemotherapeutic agent that acts on the intrinsic apoptotic pathway will be sequenced in such a way as to allow the greatest synergy between activation of the extrinsic and intrinsic apoptotic pathways of cancer cells, respectively. In particular, the present invention also relates to the order of administration of the two agents, starting with the administration of the chemotherapeutic agent which damages the DNA, and continuing with administration of the TLR3 ligand which blocks the DNA repair process. This administration sequence significantly increases the synergy of the pro-apoptotic activities of the two agents.

The present invention also relates to pharmaceutical compositions containing, with suitable excipients, separately or in a single formulation, an effective dose of a TLR3 ligand, and a chemotherapeutic agent that acts on the intrinsic apoptotic pathway. These pharmaceutical compositions are exclusively intended for humans.

Said excipients are selected according to the desired dosage form and mode of administration. Pharmaceutically acceptable excipients are well known to the person skilled in the art.

In the pharmaceutical compositions of the present invention for oral, sublingual, subcutaneous, intramuscular, intravenous, topical, intratracheal, intranasal, transdermal, rectal or intraocular administration, the active ingredients selected from TLR3 ligands and from chemotherapeutic agents that act on the intrinsic apoptotic pathway can be administered in unit dosage forms, mixed with standard pharmaceutical carriers, to animals and to humans for the prevention or the treatment of the disorders or cancers mentioned above. Suitable unit dosage forms include oral forms such as tablets, gelatin capsules, powders, granules and oral solutions or suspensions; forms for sublingual, buccal, intratracheal or intranasal administration; forms for subcutaneous, intramuscular or intravenous administration; and forms for rectal administration. For topical application, the active ingredients can be used in creams, pomades, solutions, lotions or collyria.

In order to obtain the desired prophylactic or therapeutic effect, each unit dose can contain from 0.1 mg to 10,000 mg of active ingredient in combination with a pharmaceutical carrier. This unit dose can be administered one to five times per day in order to administer a daily dose that achieves the desired effect.

When a solid composition in tablet form is prepared, the principal active ingredient is mixed with a pharmaceutical carrier, such as gelatin, starch, lactose, magnesium stearate, talc, gum arabic or analogues. The tablets can be coated with sucrose, a cellulose derivative or other suitable materials, or they can be treated so that they have extended or delayed activity and that they continuously release a predetermined quantity of the active ingredient.

A preparation in gelatin capsules is obtained by mixing the active ingredient with a diluent and then pouring the mixture obtained into soft or hard gelatin capsules.

The pharmaceutical compositions can also be provided in liquid form, for example solutions, emulsions, suspensions or syrups. Suitable liquid carriers include, for example, water and organic solvents such as glycerol or glycols, as well as mixtures of same, in varied proportions, in water.

A preparation in syrup or elixir form or for administration in the form of drops can contain the active ingredient jointly with a sweetener, preferably calorie-free, methylparaben and propylparaben as antiseptics, as well as a flavoring agent and a suitable colorant. Water-dispersible powders or granules can contain the active ingredient mixed with dispersion agents or wetting agents, or suspension agents such as polyvinylpyrrolidone, as well as sweeteners or taste correctors.

For rectal administration, suppositories prepared with binders that melt at rectal temperature, for example cocoa butter or polyethylene glycol, are used. For parenteral administration, aqueous suspensions, isotonic saline solutions or sterile and injectable solutions containing dispersion agents and/or pharmacologically compatible wetting agents, for example propylene glycol or butylene glycol, are used. The active ingredient can also be formulated in the form of microcapsules, optionally with one or more carriers or additives, or with matrices such as a polymer or a cyclodextrin (patches, extended-release forms).

The treatment combining a chemotherapeutic agent and a TLR3 ligand can also be supplemented by radiotherapy. The radiotherapy treatment can be carried out before, during or after administration of the pharmaceutical composition, and a spacing of 1 minute to 96 hours can be envisaged between the radiotherapy and the administration of the composition. The radiotherapy treatment can be any type of radiation used to treat cancer. Techniques include ionizing radiation which destroys tumor cells or damages DNA in the treatment area, notably x-rays or gamma rays or other interstitial or intracavitary brachytherapy techniques known to the person skilled in the art. Standard dosages can be used.

The experimental section below, in reference to the appended figures, illustrates the invention without being restrictive in any way.

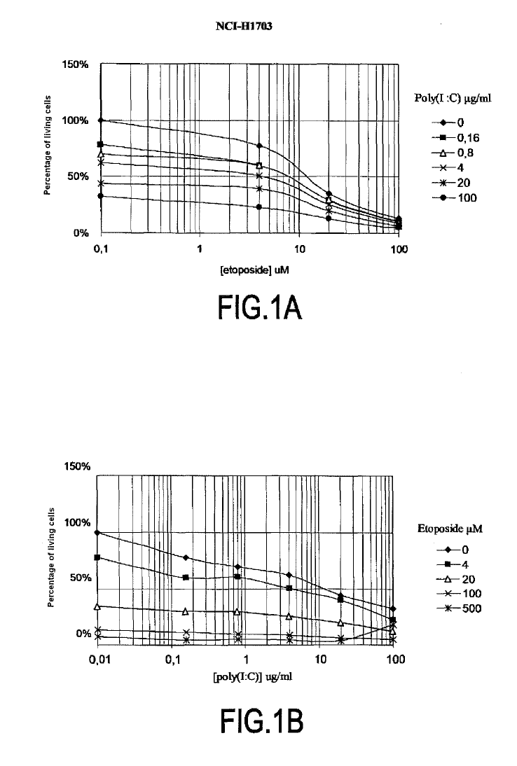

FIGS. 1A and 1B represent variations in percentages of NCIH-1703 cells alive after culture in the presence of combinations of various concentrations of etoposide and poly(I:C) in relation to untreated culture.

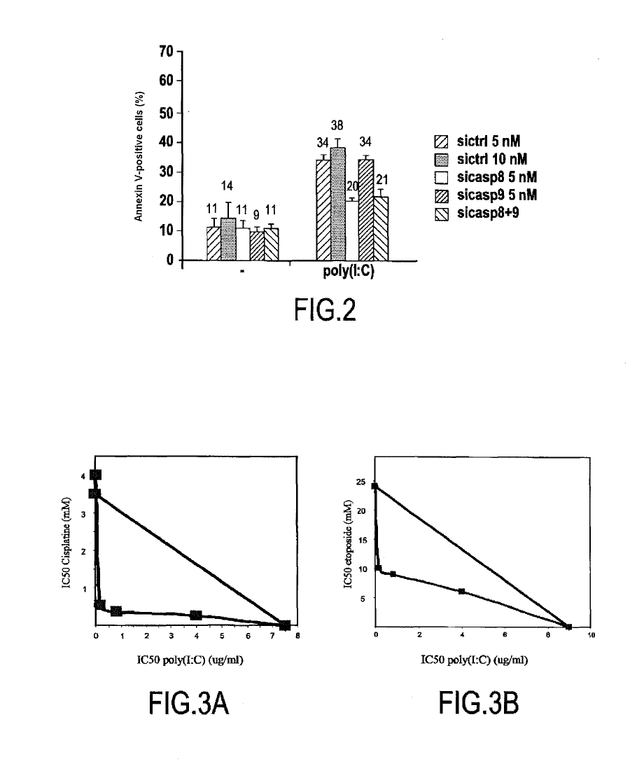

FIG. 2 shows the percentage of NCIH-H1703 cells labeled with annexin V after culture after treatment with poly(I:C), determined by flow cytometry.

FIGS. 3A and 3B represent isobolograms showing the synergistic action of poly(I:C) with cisplatin and etoposide.

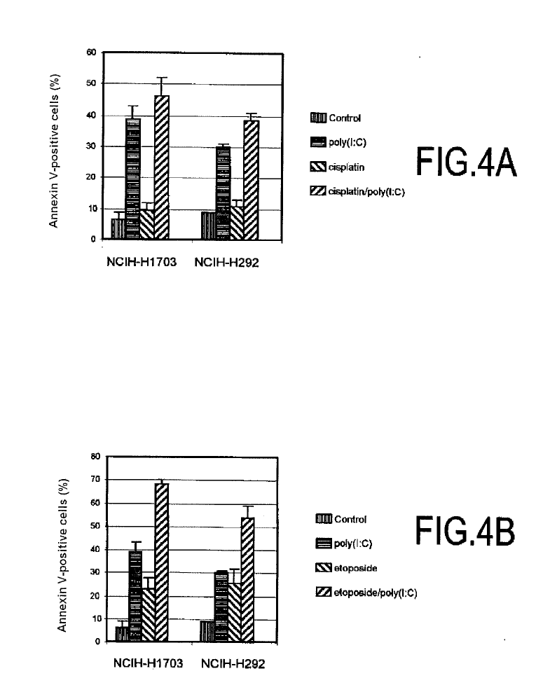

FIGS. 4A and 4B show the percentage of cells labeled with annexin V after culture after treatment with poly(I:C), etoposide and a combination of the two.

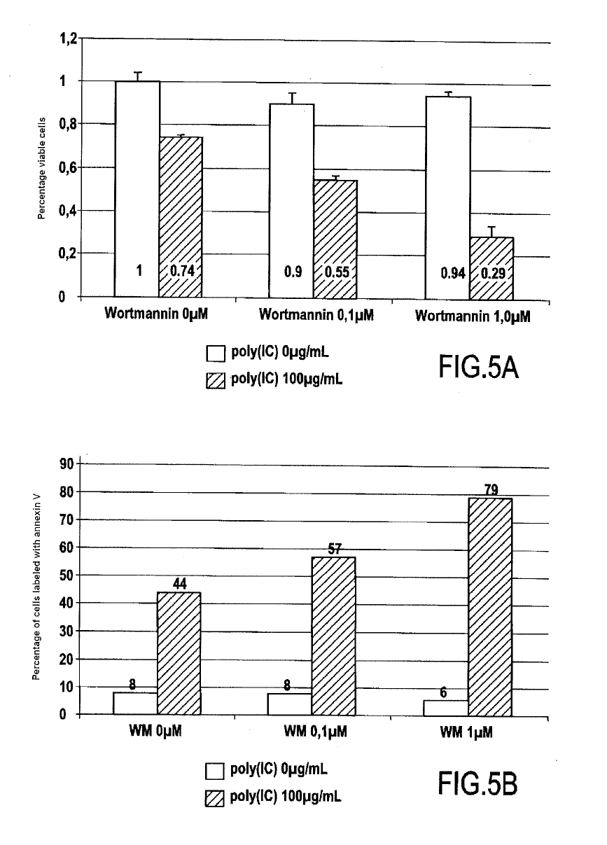

FIG. 5A represents variations in percentages of NCIH-1703 cells alive after culture in the presence of combinations of various concentrations of wortmannin and poly(I:C) in relation to the culture without poly(I:C).

FIG. 5B shows the percentage of cells labeled with annexin V after culture after treatment with wortmannin, with or without poly(I:C).

### MATERIALS AND METHODS

Reagents

Poly(I:C) was purchased from Invivogen (San Diego, Calif., USA), and trypsin (5% trypsin EDTA) and 1×DPBS were purchased from Invitrogen (Cergy Pontoise, France). Chemotherapeutic agents representing various classes were used: alkylating agents (cisplatin (Dako), oxaliplatin (Eloxatin, Sanofi-Aventis)); topoisomerase II inhibitors (etoposide (Merck), doxorubicin (Adriblastina, Pfizer)); PI3 kinase inhibitors (wortmannin, Sigma); antimetabolites (5-fluorouracil (Fluouracile, Teva), gemcitabine (Gemzar, Lilly)); the taxane family of microtubule stabilizers (paclitaxel (Taxol, Bristol Meyers), docetaxel (Taxotere, Aventis)).

Cells and General Cell Culture Conditions

NCI-H292 and NCI-H1703 are squamous cell lung cancer cell lines obtained from the American Type Culture Collection (ATCC). The cells are cultured in 100 mm-diameter dishes in complete RPMI 1640 medium with Glutabio (Eurobio Laboratories, Ulis, France) supplemented with 10% fetal calf serum (FCS) (Invitrogen, Cergy Pontoise, France) and containing 100 U/ml of penicillin (Invitrogen, Cergy Pontoise, France), 0.1 mg/ml of streptomycin (Invitrogen, Cergy Pontoise, France), 1 mM of sodium pyruvate (Invitrogen, Cergy Pontoise, France), 10 μM of HEPES (Jacques Boy Biotechnology Institute, Rheims, France). These cells are maintained at 37° C. in an atmosphere of 5% CO₂.

RNA Interference (RNAi)

The duplexes of control small interfering RNA (siRNA; Dharmacon) and siRNA specific for caspase 8 and caspase 9 (Qiagen) used are as follows: 1) control: ON-TARGETplus siCONTROL Non-Targeting siRNA #3; caspase 8 sense 5′-r(GAG UCU GUG CCC AAA UCA A)dTdT-3′, caspase 8 antisense 5′-r(UUG AUU UGG GCA CAG ACU C)dTdT-3′; caspase 9 sense 5′-r(GAG UGG CUC CUG GUA CGU U)dTdT-3′, caspase 9 antisense 5′-r(AAC GUA CCA GGA GCC ACU C)dTdT-3′. The siRNAs were transfected by the HiPerFect (Qiagen) transfection reagent according to the manufacturer's recommendations. Briefly, NCI-H1703 or NCI-H292 cells are cultured in 100 mm dishes, dislodged by trypsin, placed in 24-well plates at a concentration of 50,000 cells in 400 μl of medium per well and incubated at 37° C. during preparation of the mixes. Mixes for each well of a 24-well plate are prepared as follows: the siRNA duplexes are diluted in 100 μl of serum-free and antibiotic-free culture medium, 3 μl of HiPerFect is added, and then the solution is vortexed and incubated for 5-10 min at room temperature. 100 μl of mix is then added drop by drop to the cells and the mixture is homogenized by shaking the plate. The culture medium is changed the following day. The final siRNA concentration is 5 nM and treatment with poly(I:C) begins 72 h after transfection. For these conditions, the effectiveness of caspase 8 and caspase 9 siRNAs in decreasing the level of expression of caspase 8 and caspase 9 proteins, respectively, were measured by western blot, and reaches roughly 85% for caspase 8 siRNA and 75% for caspase 9 siRNA.

Culture Conditions for Combinations of Chemotherapy and Poly(I:C)

Cells were inoculated in 96-well plates at a concentration of 5000 cells in 100 μl of complete medium per well. The various chemotherapeutic agents were added to the decreasing final concentrations of 1 mM, 200 μM, 40 μM or 8 μM (for oxaliplatin and 5-fluorouracil) or 100 μM, 20 μM, 40 μM and 0.8 μM (for cisplatin, gemcitabine, etoposide, doxorubicin, paclitaxel and docetaxel). After 2 h of incubation at 37° C., the culture medium was aspirated and replaced with 100 μl of complete medium containing decreasing concentrations of poly(I:C) (100 μg/ml, 20 μg/ml, 4 μg/ml, 0.8 μg/ml); the cells were then cultured for 70 h. Each treatment condition was carried out in duplicate. The results are expressed as the relative number of viable cells in relation to the untreated control cultures.

Analysis of Number of Living Cells

The analysis was carried out using the CellTiter 96° AQᵤₑₒᵤₛ One Solution Cell Proliferation Assay kit (Promega, Charbonnière, France) according to the manufacturer's instructions. Briefly, 20 μl of MTS was added to the culture medium of each well. The cells were placed in an incubator for 2 h at 37° C. Absorbance at 490 nm was analyzed using a spectrophotometer (Multiskan® EX; Thermo Fisher Scientific). A second measurement at 690 nm was made to exclude nonspecific absorbance. The baseline optical density (blank) represents the average of three wells containing the culture medium alone and was subtracted from the recorded values. Each value represents the average OD of the duplicates. The results are expressed as relative OD values in relation to the untreated control cultures.

Annexin V-FITC/Propidium Iodide Labeling

The cells are inoculated in 24-well plates at a concentration of 3.5-10⁴ cells per well. After 48 h, the culture medium is replaced at various times by culture medium alone or culture medium containing 100 μg/ml of poly(I:C). The supernatant is recovered and the cells are rinsed with Dulbecco's phosphate buffered saline (DPBS). As before, the supernatant is recovered and the cells are treated with trypsin. Once the cells are dislodged, trypsin action is stopped with culture medium. The contents of the well are recovered and mixed with the supernatants previously collected. The cells are centrifuged (1400 rpm, 5 min) and the supernatant is withdrawn. Annexin V-FITC/propidium iodide labeling is carried out using an Annexin V-FITC kit (AbCys SA, Paris, France) according to the manufacturer's instructions. Briefly, the cells are suspended in 100 μl of binding buffer and then incubated with 2.5 μl of annexin V for 10 to 15 minutes away from light at room temperature. A sufficient volume of propidium iodide is added to obtain a final concentration of 1 μg/ml. The samples are analyzed with a FACSCalibur flow cytometer (BD Bioscience, San Jose, Calif., USA) and the data are treated using the FlowJo software (TreeStar, San Carlos, Calif., USA).

Mathematical Analysis of the Effects of the Combination of Chemotherapeutic Agent and Poly(I:C) on Cell Survival

The 50% inhibitory concentrations (IC₅₀s), defined as the concentrations necessary to obtain 50% fewer cells than the untreated control after culture, were determined for each molecule used alone or in combination. The IC₅₀s represent the averages of all the experiments performed. To mathematically determine the nature of the interaction between the two molecules, two complementary methods were used: calculation of the combination index (CI) (Chou et al.: Quantitative analysis of dose-effect relationships: the combined effects of multiple drugs or enzyme inhibitors, Adv Enzyme Regul 1984, 22:27-55) and construction of an isobologram (Steel et al.: Exploitable mechanisms in combined radiotherapy-chemotherapy: the concept of additivity, Int J Radiat Oncol Biol Phys 1979, 5:85-91).

The IC₅₀ chemotherapy unit was homogenized to μg/ml. Together, all of these IC₅₀s can be used to calculate combination indexes (CIs), defined by the equation (IC_{50(chemotherapy/poly(I:C))}/IC_{50(chemotherapy))}+(IC_{50(poly(I:C)/chemotherapy)})/IC_{50(poly(I:C))})+(IC_{50(chemotherapy/poly(I:C))}×IC_{50(poly(I:C)/chemotherapy)})/(IC_{50(chemotherapy)}×IC_{50(poly(I:C))})), where IC_{50 (chemotherapy)} and IC_{50(poly(I:C))} respectively represent IC₅₀s of chemotherapy and of poly(I:C) used alone, and IC_{50(chemotherapy/poly(I:C))} and IC_{50(poly(I:C)/chemotherapy)} respectively represent IC₅₀s of chemotherapy and poly(I:C) used in combination. The average of all the CIs is calculated in order to obtain the average CI that determines the nature of the interaction between the two molecules, according to its value: a CI of 0.1 to 0.9 indicates synergy; a CI of 0.9 to 1.1 indicates additivity; a CI of 1.1 to 10 indicates antagonism.

Interpretation of CI values (Chou et al., 1984)

| | |
|---|---|
| 0.3-0.7 | Synergy |
| 0.7-0.85 | Moderate synergy |
| 0.85-0.9 | Weak synergy |
| 0.9-1.1 | Additivity |
| 1.1-1.2 | Weak antagonism |
| 1.2-1.45 | Moderate antagonism |
| 1.45-3.3 | Antagonism |

To build the isobolograms, the abscissa and the ordinate represent the IC₅₀s of poly (I:C) and of the chemotherapeutic agent, respectively. The IC₅₀ of poly(I:C) used alone is plotted on the x-axis and the IC₅₀ of the chemotherapeutic agent used alone is plotted on the y-axis. A line connects these two points: it represents the theoretical line of additivity. The IC_{50(chemotherapy)/poly(I:C))} and IC_{50(poly(I:C)/chemotherapy)} values are plotted on the graph. These points are connected and constitute a curve having as extremities the intersections between the line of additivity and the x-axis and the y-axis. If the curve is confounded with, or very near to the line of additivity, additivity between poly(I:C) and the chemotherapeutic agent is identified. If the curve is below or above, the two molecules act synergistically or antagonistically, respectively.

Cultures Conditions for Combinations of Radiotherapy and Poly(I:C)

Cells are inoculated one day before in T25 (25 cm²) culture flasks at a concentration of 1-10⁶ cells/flask. After 24 h, the culture medium is replaced with simple culture medium or medium containing poly(I:C) (10 μg/ml). One hour after the change of medium, the cells receive various doses of radiation (2 Gy, 5 Gy, 10 Gy). After 24 h, the cells are labeled using the Annexin V-FITC/propidium iodide kit as described above.

Results

Etoposide and Poly(I:C) have a Complementary Effect In Vitro on the Reduction of the Number of Living Lung Cancer Cells

Variations in percentages of NCIH-1703 cells alive after culture in the presence of combinations of various concentrations of etoposide and poly(I:C) in relation to the untreated culture are presented in FIGS. 1A and 1B. FIGS. 1A and 1B represent the percentage of living cells after culture as a function of (A) etoposide concentration or (B) poly(I:C) concentration, respectively. Each figure represents six experiments carried out independently.

It is observed that the effect of an intermediate concentration of poly(I:C) alone (e.g., 4 μg/ml), which reduces the number of living cells by ˜35%, is doubled (˜70% reduction) by pre-incubation for 2 h with 20 μM of etoposide (FIG. 1A). Conversely, etoposide alone at a concentration of 4 μM results in a loss of approximately 25% of viable cells, and this reduction increases to ˜60% after the addition of 60 μg/ml of poly(I:C) (FIG. 1B).

Poly(I:C) Induces Apoptosis of Cancer Cell Lines by Activating the Extrinsic Pathway

In relation to the mechanism of reduction of the number of living cells, the percentage of cells labeled with annexin V after culture after treatment with poly(I:C) was determined by flow cytometry. FIG. 2 shows the effect of transfection of caspase 8 and caspase 9 siRNAs on the percentage of annexin V-positive cells induced by treatment with poly(I:C). Seventy-two hours after transfection of the control (sictrl), caspase (sicasp8), caspase 9 (sicasp9) or caspase 8+caspase 9 (sicasp8+9) siRNAs, NCI-H1703 cells are treated with 100 μg/ml of poly(I:C) for 24 h or are not treated. The percentage of annexin V-positive cells is measured by flow cytometry. The results presented are the average of three experiments carried out independently. Error Bar, ±SE.

FIG. 2 shows that ˜⅓ of the NCI-H1703 cells are in apoptosis 24 h after exposure to TLR3 ligand (compared to 11% in the control wells). Inhibition of caspase 8 expression (by transfection of a specific siRNA) significantly decreases the percentage of apoptotic cells (˜20%), whereas suppression of caspase 9 expression has no significant effect on apoptosis. Similar results were obtained with the NCI-H292 line. It thus appears that poly(I:C) induces apoptosis of the lung cancer cell lines analyzed, and that this apoptosis occurs by activation of the extrinsic apoptotic pathway.

Poly(I:C) has a Synergistic Effect with Numerous Chemotherapeutic Agents

Comparison of the IC₅₀ of poly(I:C) used alone or in combination with various molecules representing the four principal classes of chemotherapeutic agents makes it possible to calculate a combination index (CI) whose value can represent synergy (CI<1), additivity (CI˜1) or antagonism (CI>1). The CI values calculated for the various chemotherapeutic agents, presented in table 1 below, show strong synergy on NCI-H1703 and NCI-H292 cell lines with platinum-derived alkylating agents (cisplatin and oxaliplatin) and topoisomerase II inhibitors (etoposide and doxorubicin). With regard to 5-fluorouracil, paclitaxel and docetaxel, moderate synergy is observed. For gemcitabine, the results show additivity without synergy for NCI-H1703 and antagonism for NCI-H292.

TABLE 1

| | | NCI-H1703 | NCI-H292 |
|---|---|---|---|
| Poly (I:C) | IC₅₀ | 19.9 μg/ml | 14.3 μg/ml |
| | | (±3.23 μg/ml) | (±2.09 μg/ml) |

Platinum-derived alkylating agents

| | | | |
|---|---|---|---|
| Cisplatin | | (n = 5) | (n = 5) |
| | IC₅₀ | 1.1 μM ± 0.13 μM | 3.4 μM ± 0.8 μM |
| | CI | 0.62 ± 0.03 | 0.50 ± 0.08 |
| Oxaliplatin | | (n = 3) | (n = 3) |
| | IC₅₀ | 170.0 μM ± 67.57 μM | 123.3 μM ± 43.33 μM |
| | CI | 0.67 ± 0.177 | 0.63 ± 0.19 |

Topoisomerase II inhibitors

| | | | |
|---|---|---|---|
| Etoposide | | (n = 6) | (n = 3) |
| | IC₅₀ | 32.67 μM ± 10.56 μM | 18.67 μM ± 6.96 μM |
| | CI | 0.59 ± 0.06 | 0.68 ± 0.11 |
| Doxorubicin | | (n = 3) | (n = 3) |
| | IC₅₀ | 3.73 μM ± 2.14 μM | 1.03 μM ± 0.3 μM |
| | CI | 0.72 ± 0.06 | 0.62 ± 0.08 |

Antimetabolites

| | | | |
|---|---|---|---|
| Gemcitabine | | (n = 2) | (n = 6) |
| | IC₅₀ | 0.9 μM ± 0.6 μM | 0.6 μM ± 0.14 μM |
| | CI | 1.0 ± 0.07 | 1.3 ± 0.24 |
| 5-Fluouracile | | | (n = 3) |
| | IC₅₀ | | 133 μM ± 12 μM |
| | CI | | 0.78 ± 0.09 |

Microtubule depolymerization inhibitors

| | | | |
|---|---|---|---|
| Paclitaxel | | (n = 3) | (n = 3) |
| | IC₅₀ | 0.12 μM ± 0.09 μM | 0.21 μM ± 0.14 μM |
| | CI | 0.83 ± 0.012 | 0.71 ± 0.0064 |
| Docetaxel | | | (n = 3) |
| | IC₅₀ | | 10.6 μM ± 9.7 μM |
| | CI | | 0.68 ± 0.2 |

Isobolographic Analysis Illustrates the Synergy of the Combination of Poly(I:C) with Cisplatin and Etoposide on NCI-H292 and NCI-1703 cell lines

The IC₅₀s of cisplatin, etoposide and poly(I:C) used alone with NCI-H292 cells are 3.4 μM (±0.8 μM), 18.67 μM (±6.96 μM) and 14.3 μg/ml (±2.09 μg/ml), respectively (table 1). The isobolograms, which represent the average of six experiments carried out independently, show that the number of NCI-H292 cells alive after culture are decreased by 50% by combining cisplatin at a concentration of ˜0.5 mM with poly(I:C) at a concentration of ˜0.5 μg/ml (FIG. 3A), or etoposide at a concentration of ˜8 mM with poly(I:C) at a concentration of ˜2 μg/ml (FIG. 3B). The isobolograms illustrate the synergistic action of poly(I:C) with cisplatin and etoposide. Similar results were obtained for the NCI-H1703 cell line.

The Pro-Apoptotic Activities of Poly(I:C) and Cisplatin or Etoposide on NSCLC Cell Lines are Additive

Poly(I:C) induces apoptosis of NSCLC NCIH-1703 and NCIH-292 cell lines. To determine whether the greater reduction in the number of cells alive after treatment with the combination of poly(I:C) and cisplatin or etoposide resulted at least partially from an increase in apoptosis, the cells were labeled with annexin V after 24 h of culture as described in the “Materials and methods” section above. The concentrations of poly(I:C) and of the chemotherapeutic agents were adjusted to correspond to the IC₅₀ of each cell line. It is observed for the two cell lines that a brief (2 h) exposure of the cells to cisplatin before the addition of TLR3 ligand increases the percentage of apoptotic cells, but this increase is statistically significant only for the NCIH-292 cell line (FIG. 4A). With respect to etoposide, a significant additive effect is observed for both cell lines (FIG. 4B). It thus appears that the additive pro-apoptotic effects of the chemotherapeutic agents and poly(I:C) after 24 h participate in the strong synergy observed in terms of the number of cells alive after 72 h of culture.

Combination of PI3K Inhibitor and TLR3 Ligand

Similarly, a study was undertaken to determine the viability of cells of the NCI-H1703 human squamous cell lung cancer cell line cultured for 24 h in the presence of TLR3 ligand (poly(I:C), 100 μg/ml) at various concentrations (0 μM, 0.1 μM and 1.0 μM) of wortmannin, a specific PI3 kinase inhibitor. The results are presented in FIG. 5A.

In the same way, a study was undertaken to determine the percentage of cells of the NCI-H1703 human squamous cell lung cancer cell line in apoptosis (FACS analysis after labeling with annexin V-FITC/propidium iodide) after culture for 24 h in the presence of TLR3 ligand (poly(I:C), 100 μg/ml) with various concentrations (0 μM, 0.1 μM and 1.0 μM) of specific PI3 kinase inhibitor, wortmannin (WM). The results are presented in FIG. 5B. It appears that the combination of poly(I:C) and wortmannin increases, in a synergistic manner, pro-apoptotic activity on cancer cells, compared with each compound used separately. Moreover, the synergy is strong: with 1 μM wortmannin alone, 6% fewer living cells are observed after 24 h; with 100 μg/ml poly(I:C) alone, 26% fewer living cells are observed after 24 h; when both molecules are used in combination at these same concentrations, however, 71% fewer living cells are observed after 24 h.

1. A drug comprising separately or together (i) a TLR3 ligand and (ii) a chemotherapeutic agent that acts on the intrinsic apoptotic pathway, for simultaneous or sequential administration in the treatment of cancer, wherein the chemotherapeutic agent is selected from topoisomerase II inhibitors, platinum-derived alkylating agents and PI3 kinase inhibitors.

2. The drug according to claim 1, wherein said drug is intended for successive administration of (i) an agent that acts on the intrinsic apoptotic pathway and then (ii) a TLR3 ligand in the treatment of cancer.

3. The drug according to claim 1, wherein said drug it is intended for the treatment of squamous cell lung cancer, colon adenocarcinoma, mesothelioma, glioma, breast adenocarcinoma, melanoma, clear cell kidney cancer, prostate cancer, hepatocellular carcinoma or multiple myeloma.

4. The drug according to claim 1, wherein the chemotherapeutic agent is a platinum-derived alkylating agent.

5. The drug according to claim 4, wherein the platinum-derived alkylating agent is selected from cisplatin and oxaliplatin.

6. The drug according to claim 1, wherein the chemotherapeutic agent is a topoisomerase II inhibitor.

7. The drug according to claim 6, wherein the topoisomerase II inhibitor is selected from etoposide and doxorubicin.

8. The drug according to claim 1, wherein the chemotherapeutic agent is a PI3 kinase inhibitor.

9. The drug according to claim 8, wherein the PI3 kinase inhibitor is selected from wortmannin, LY294002, PIK-90/BAY2-47, XL765, XL147, SF1126, NVP-BEZ235, NVP-BGT226, GDC-0941, CAL-101 and GSK1059615.

10. The drug according to claim 1, wherein said drug is provided as a single pharmaceutical composition that combines, in the same formulation, (i) a TLR3 ligand and (ii) a chemotherapeutic agent that acts on the intrinsic apoptotic pathway.

11. The drug according to claim 1, wherein the TLR3 ligand is a synthetic double-stranded RNA.

12. The drug according to claim 1, wherein the TLR3 ligand is a TLR3 agonist.

13. The drug according to claim 1, wherein the TLR3 ligand is poly(I:C).

14. The drug according to claim 1, wherein the TLR3 ligand is a specific TLR3 ligand such as poly(A:U).

15. Use of a TLR3 ligand and an agent that acts on the intrinsic apoptotic pathway for the preparation of the drug according to claim 1.
